(19) United States
(12) Patent Application Publication
(10) Pub. No.: US 2024/0139142 A1
(43) Pub. Date: May 2, 2024

(54) METHOD FOR PREVENTING OR TREATING LIVER DISEASE

(71) Applicants: TAIPEI VETERANS GENERAL HOSPITAL, Taipei City (TW); NATIONAL YANG MING CHIAO TUNG UNIVERSITY, Hsinchu (TW); PHARMAESSENTIA CORPORATION, Taipei City (TW)

(72) Inventors: Jaw-Ching WU, Taipei City (TW); Yung-Sheng CHANG, Hsinchu (TW); Kuo-Hsi KAO, Taipei City (TW); Chan-Kou HWANG, Taipei City (TW); Ko-Chung LIN, Taipei City (TW)

(73) Assignees: TAIPEI VETERANS GENERAL HOSPITAL, Taipei City (TW); NATIONAL YANG MING CHIAO TUNG UNIVERSITY, Hsinchu (TW); PHARMAESSENTIA CORPORATION, Taipei City (TW)

(21) Appl. No.: 18/467,437

(22) Filed: Sep. 14, 2023

(30) Foreign Application Priority Data
Oct. 14, 2022 (TW) 111139101

(51) Int. Cl. A61K 31/26; A61K 45/06; A61P 1/16; A61P 35/00

(52) U.S. Cl. CPC A61K 31/26; A61K 45/06; A61P 1/16; A61P 35/00

(57) ABSTRACT

Provided is a method for preventing or treating a liver disease, including administering a therapeutically effective amount of pharmaceutical composition to a subject in need, and the pharmaceutical composition includes the isothiocyanate structural modified compound and a pharmaceutically acceptable carrier thereof.

### TECHNICAL FIELD

The present disclosure relates to a method for preventing or treating liver disease. More particularly, it relates to a method for preventing or treating hepatitis, abnormal lipid metabolism of liver/steatosis, abnormal carbohydrate metabolism of liver, liver fibrosis, liver cirrhosis or liver hepatocellular carcinoma (HCC).

### BACKGROUND

Cancer is the first leading cause of death among the R.O.C. citizens, and the second causes of cancer death are liver hepatocellular carcinoma and intrahepatic cholangiocarcinoma, wherein more than 7,000 people died of liver hepatocellular carcinoma every year. Also, liver hepatocellular carcinoma is the third cause of the cancer death worldwide, wherein more than 500,000 patients died of liver hepatocellular carcinoma every year. It is known that hepatitis, liver cirrhosis, and liver hepatocellular carcinoma are “the trilogy of liver disease.” Chronic hepatitis virus infection, including the hepatitis B virus (HBV) infection, is one of the main risk factors leading to the subsequent liver cirrhosis or liver hepatocellular carcinoma. Until now, only 20% to 30% of the liver hepatocellular carcinoma patients are able to be treated by surgery, and the recurrence rate of these patients is still high 5 years after the surgery.

In the liver hepatocellular carcinoma medications, Sorafenib (Nexavar) is one of the small-molecule targeted drugs being approved for treating liver hepatocellular carcinoma. It is a multitargeted kinase inhibitor (mitogen-activated protein kinase, MAPK; extracellular signal-regulated kinase, ERK) inhibiting the proliferation, invasion and metastasis of tumor cells by inhibiting the MAPK signal transduction of hepatocellular carcinoma. However, it can only extend patients' lifespan for about 3 months. Moreover, the drug resistance of Sorafenib in liver hepatocellular carcinoma is very common, and there is a high percentage of liver hepatocellular carcinoma patients still having situations of increased growth, recurrence or metastasis of tumors after long-term treatment of Sorafenib. Besides, Nexavar is also reported to have stronger side effects.

There are many molecules that participate in the mechanisms of drug resistance of Nexavar in liver hepatocellular carcinoma, e.g. overactivation of the signaling transduction pathways of PI3K/Akt and RAF/MEK/ERK; additionally, transforming growth factor beta (TGF-β), epidermal growth factor receptor (EGFR), hypoxia-inducible factor 1-alpha (HIF-1α), HIF-2α, pregnane X receptor (PXR), long noncoding RNAs (lncRNAs), microRNAs and vascular endothelial growth factor (VEGF) and different genotypes and phenotypes of the receptors thereof (VEGFR), are all related to the development of drug resistance.

However, the related molecules described above still have many limitations when applying in the prediction of drug resistance of liver hepatocellular carcinoma. For example, signaling transduction pathways of PI3K/Akt and RAF/MEK/ERK also participate in many normal physiological regulation in normal cells, and thus lacking specificity in diagnosis; the expression of different genotypes of VEGF and receptor thereof (VEGFR) is limited by the difference between individual patients and tumor microenvironment, which cannot be used as the biomarker reflecting the drug resistance accurately. Although the microRNA has specificity to a certain degree, it is still difficult to be applied broadly due to the limitation of current detection techniques.

To date, there is still a lack of effective and safe drugs for treating liver diseases mediated by pathways of EMT, ROS-deposit or abnormal autophagy, such as hepatitis; abnormal lipid metabolism of liver/steatosis; liver fibrosis; liver cirrhosis; and/or liver hepatocellular carcinoma, particularly to those liver hepatocellular carcinoma patients, who have already developed drug resistance, there is still a lack of effective second-line medication. In view of a serious threat of liver hepatocellular carcinoma to human health, developing new drugs for liver diseases and therapeutic regimen for liver hepatocellular carcinoma, as well as biomarkers for liver hepatocellular carcinoma drug resistance are thus crucial and emergent problem currently.

### SUMMARY

According to the problems described above, the present disclosure provides a use of a isothiocyanate structural modified compound in the manufacture of a pharmaceutical composition for preventing or treating a liver disease in a subject, wherein the pharmaceutical composition includes the isothiocyanate structural modified compound and a pharmaceutically acceptable carrier thereof.

The present disclosure also provides a method for preventing and treating liver diseases, including administering a therapeutically effective amount of pharmaceutical composition to a subject in need, wherein the pharmaceutical composition comprises an isothiocyanate structural modified compound and a pharmaceutically acceptable carrier thereof.

In at least one embodiment of the present disclosure, the isothiocyanate structural modified compound is PCH1152, shown as formula (I) below:

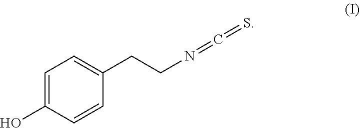 [SMILES: Oc1ccc(CCN=C=S)cc1]

In at least one embodiment of the present disclosure, the isothiocyanate structural modified compound is P1130, shown as formula (II) below:

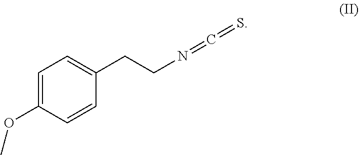

In at least one embodiment of the present disclosure, the liver diseases are hepatitis, abnormal lipid metabolism of liver/steatosis, abnormal carbohydrate metabolism of liver, liver fibrosis, liver cirrhosis and/or liver hepatocellular carcinoma. In some embodiments of the present disclosure, the liver hepatocellular carcinoma is liver hepatocellular carcinoma with drug resistance.

In at least one embodiments of the present disclosure, the liver diseases result from epithelial-mesenchymal transition, excess reactive oxygen species, anti-apoptosis and/or abnormal autophagy, etc.

In some embodiments of the present disclosure, the pharmaceutical composition comprises isothiocyanate structural modified compound PCH1152 as the only active ingredient for preventing or treating liver related diseases in a subject. In the other embodiments of the present disclosure, the pharmaceutical composition comprises isothiocyanate structural modified compound P1130 as the only active ingredient for preventing or treating liver related diseases in a subject

In at least one embodiments of the present disclosure, the prevention or treatment comprises promoting apoptosis of liver hepatocellular carcinoma cells, reducing expressions of the liver hepatocellular carcinoma-related molecules, reducing the incidence of liver hepatocellular carcinoma, inhibiting the growth and invasion of the liver hepatocellular carcinoma, reducing the metastasis of the liver hepatocellular carcinoma, reducing the recurrence of the liver hepatocellular carcinoma and/or reducing the drug resistance of the liver hepatocellular carcinoma.

In at least one embodiment of the present disclosure, the prevention or treatment comprises maintaining the normal hepatic function.

In at least one embodiment of the present disclosure, the prevention or treatment comprises increasing the sensitivity of the liver hepatocellular carcinoma to an anti-liver hepatocellular carcinoma drug.

In at least one embodiment of the present disclosure, the prevention or treatment comprises reducing or inhibiting the expressions of the following liver diseases/liver hepatocellular carcinoma-related molecules: multidrug resistance-associated protein (MRP) ABCC1, multidrug resistance-associated protein ABCC2, Ubiquitin-specific peptidase 22 (USP22), epithelial-mesenchymal transition (EMT) regulatory protein Twist, epithelial-mesenchymal transition regulatory protein Snail, anti-apoptosis protein Mcl-1 (myeloid cell leukemia-1) and/or reactive oxygen species.

In at least one embodiment of the present disclosure, the pharmaceutical composition is administered orally, intravenously, enterally or subcutaneously to the subject.

In at least one embodiment of the present disclosure, the pharmaceutical composition is administered to the subject for at least 1 day, at least 2 days, at least 3 days, at least 4 days, at least 5 days, at least 1 week, at least 2 weeks, at least 3 weeks, at least 4 weeks, at least 5 weeks or at least 6 weeks. In the other embodiments, the pharmaceutical composition is administered to the subject for at least 1 month, at least 2 months, at least 3 months, at least 4 months, at least 5 months or at least 6 months.

In at least one embodiment of the present disclosure, the pharmaceutical composition is administered in combination with anticancer drug. In some embodiments of the present disclosure, the pharmaceutical composition is administered in combination with Sorafenib (Nexavar), Doxorubicin (Adriamycin) or Regorafenib (Stivarga).

In at least one embodiment of the present disclosure, the pharmaceutical composition is administered in combination with a targeted therapy drug, an immunotherapy drug, or a radiotherapy drug.

In at least one embodiment of the present disclosure, the pharmaceutical composition is administered in combination with a metabolic disease drug, a cardiovascular disease drug or an endocrine disease drug.

In at least one embodiment of the present disclosure, the pharmaceutical composition is administered in combination with a drug for blood glucose control or a drug for blood lipid control.

In some embodiments of the present disclosure, the pharmaceutical composition is administered with Metformin or Statin-like drug.

In at least one embodiment of the present disclosure, the pharmaceutical composition is administered in combination with anticancer drug simultaneously, sequentially or separately. In the other embodiments of the present disclosure, the pharmaceutical composition is administered in combination with a metabolic disease drug, a cardiovascular disease drug or an endocrine disease drug simultaneously, sequentially or separately.

In the other aspect, the present disclosure provides a method for preventing or treating liver hepatocellular carcinoma in a subject, comprising administering a therapeutically effective amount of pharmaceutical composition of the isothiocyanate structural modified compound PCH1152 or P1130 and a pharmaceutically acceptable carrier thereof to the subject.

In at least one embodiment of the present disclosure, the method further comprises administrating an additional therapy to the subject, for example, but not limit to a targeted therapy, an immunotherapy, a radiotherapy and/or a surgical therapy.

In at least one embodiment of the present disclosure, the method is used for treating subject with drug resistance to anticancer drug. In some embodiments of the present disclosure, the method is used for treating subject with drug resistance to Sorafenib; in the other embodiments of the present disclosure, the method is used for treating subject with drug resistance to Regorafenib; still in the other embodiments of the present disclosure, the method is used for treating subject with drug resistance to Doxorubicin.

In at least one embodiment of the present disclosure, the drug resistance to anticancer drug is evaluated by taking the expressions of USP22 and/or ABCC1 in the subject as biomarkers, wherein the expression of USP22 is positively correlated with the expression of ABCC1. In some embodiments, the anticancer drug is Sorafenib (Nexavar), Doxorubicin (Adriamycin) or Regorafenib (Stivarga).

### BRIEF DESCRIPTION OF THE DRAWINGS

FIG. 1 shows the effect of the isothiocyanate structural modified compound of the present disclosure on the apoptosis in the SNU-449 liver hepatocellular carcinoma cell line. DMSO: control group, PEITC: natural isothiocyanate compound, P1130 and PCH1152: isothiocyanate structural modified compound.

FIG. 2 shows the inhibiting effect of the isothiocyanate structural modified compound of the present disclosure on the expressions of the multidrug resistance-associated protein ABCC2, epithelial-mesenchymal transition regulatory protein Twist, and anti-apoptosis protein Mcl-1 in the SNU-449 and Mahlavu liver hepatocellular carcinoma cell line. DMSO: control group; P1130, PCH1152 and PCH1168: isothiocyanate structural modified compound.

FIG. 3A and FIG. 3B show that isothiocyanate structural modified compound of the present disclosure has selectivity to inhibit liver hepatocellular carcinoma growth. FIG. 3A shows the results of cell growth of SNU-449 liver hepatocellular carcinoma cell line and normal liver cell line THLE-2 detected by MTS assay after being treated with isothiocyanate structural modified compound. FIG. 3B shows the results of SNU-449 liver hepatocellular carcinoma cell line and normal liver cell line THLE-2 analyzed by western blot after being treated with isothiocyanate structural modified compound.

FIG. 4A to FIG. 4E show the inhibiting effect of isothiocyanate structural modified compound PCH1152 on liver tumor growth in the animal. FIG. 4A shows the weight change of the mice in the control group and the experimental group. FIG. 4B shows the comparison of the food intake per day of the mice in the control group and the experimental group. FIG. 4C shows the comparison of the alanine transaminase (ALT) value of the mice in the control group and the experimental group. FIG. 4D shows the change of the subcutaneous tumor volume of the mice in the control group and the experimental group. FIG. 4E shows the results of the protein expressions in subcutaneous tumor of the mice analyzed by western blot.

FIG. 5A and FIG. 5B show the ability of isothiocyanate structural modified compound PCH1152 of the present disclosure to remove reactive oxygen species. FIG. 5A shows the clearance (detected by DHE fluorescence) of PCH1152 alone or combining with NAC (N-acetylcysteine) on Antimycin A-induced ROS after treating LX-2 cell with PCH1152 alone or combining with NAC for 24 hours, with or without the presence of Antimycin A. FIG. 5B shows the clearance (detected by DHE fluorescence) of PCH1152 alone, NAC alone or both on Hepatitis B virus-induced ROS in the human liver hepatocellular carcinoma cell line Huh-7, wherein the Huh-7 cells were transfected by the expression plasmid (pGEM C12) of Hepatitis B virus C12 or not, and without any treatment or treated with PCH1152 alone, NAC alone or both.

FIG. 6A to FIG. 6H show the changes of the body weight, food intake, water intake, tumor incidence, tumor weight and volume; and the ALT (alanine aminotransferase) value of the Hepatitis B virus transgenic mouse under long-term treatment of the isothiocyanate structural modified compound PCH1152 of the present disclosure. FIG. 6A shows the structural stability of the PCH1152 in the drinking water for the mice. FIG. 6B shows the body weight changes of the mice in the control group and the experimental group. FIG. 6C shows the changes of the daily food intake of the mice in the control group and experimental group. FIG. 6D shows the changes of the daily water intake of the mice in the control group and experimental group. FIG. 6E shows the comparison of the mouse liver tumor incidence rate in the control group and the experimental group. FIG. 6F shows the time point when ALT in the mouse serum of the control group and the experimental group. FIG. 6G and FIG. 6H show the comparison of the mouse liver tumor weight and volume in the control group and the experimental group, respectively.

FIG. 7 shows the size of the region of the lipid deposition or fibrosis in the mouse liver tissue stained with Oil Red or Sirius Red.

FIG. 8A and FIG. 8B show the comparing results of the expression of the autophagy-related P62 protein and the LC3B II to LC3B I ratio in the liver tissue of mice (with or without liver hepatocellular carcinoma) in the PCH1152-treated group and the control group (Hsc70 as an internal control); the liver tissue is analyzed by western blot.

FIG. 9A and FIG. 9B show the synergistic effect of using isothiocyanate structural modified compound PCH1152 of the present disclosure in combination with anticancer drug Regorafenib or Doxorubicin. FIG. 9A shows the effect of using PCH1152 in combination with anticancer drug Regorafenib or Doxorubicin in the Mahlavu liver hepatocellular carcinoma cell line. FIG. 9B shows the effect of using PCH1152 in combination with anticancer drug Regorafenib or Doxorubicin in the SNU-449 liver hepatocellular carcinoma cell line.

FIG. 10A to FIG. 10D show the synergistic effect of using isothiocyanate structural modified compound PCH1152 of the present disclosure in combination with metabolic disease drug Statin or Metformin. FIG. 10A and FIG. 10B show the inhibiting effect of PCH1152 in combination with statins (Simvastatin, Lovastatin and/or Atorvastatin) or Metformin on liver hepatocellular carcinoma cell line growth, respectively. FIG. 10C and FIG. 10D show the inhibiting effect of PCH1152 in combination with statins (Simvastatin, Lovastatin and/or Atorvastatin) or Metformin on SNU-449 liver hepatocellular carcinoma cell line growth, respectively.

FIG. 11 shows the protein expression levels of the phosphorylated Akt and phosphorylated Erk in Sorafenib-drug resistance-liver hepatocellular carcinoma cell line. WT: wild-type liver hepatocellular carcinoma cell line. R: Nexavar-drug resistance-liver hepatocellular carcinoma cell line.

FIG. 12 shows the expression levels of ubiquitin-specific peptidase family and ABCC1 multidrug resistance-associated protein in Nexavar-drug resistance-liver hepatocellular carcinoma cell line. WT: wild-type liver hepatocellular carcinoma cell line. R: Nexavar-drug resistance-liver hepatocellular carcinoma cell line

FIG. 13A to FIG. 13B show the effect of inhibiting endogenous USP22 expression in Nexavar-drug resistance-liver hepatocellular carcinoma cell line by RNA interference (RNAi) technique on ABCC1 and ABCC2 expression levels. FIG. 13A shows the protein expression of each gene analyzed by western blot. FIG. 13B shows the mRNA expression of each gene analyzed by real-time quantitative polymerase chain reaction (qPCR). WT: wild-type liver hepatocellular carcinoma cell line. R: Nexavar-drug resistance-liver hepatocellular carcinoma cell line.

FIG. 14 shows the effect of isothiocyanate structural modified compound PCH1152 and PCH1130 of the present disclosure on liver hepatocellular carcinoma-related protein molecule expression in Nexavar-drug resistance-liver hepatocellular carcinoma cell line and apoptosis. WT: wild-type liver hepatocellular carcinoma cell line. R: Nexavar-drug resistance-liver hepatocellular carcinoma cell line.

FIG. 15A and FIG. 15B show the USP22 and ABCC1 multidrug resistance-associated protein expressions and the correlation thereof in tissue of the liver hepatocellular carcinoma removed from the patients with Nexavar drug resistance by surgery.

### DETAILED DESCRIPTION

The following embodiments are provided to illustrate the present disclosure in detail. A person having ordinary skill in the art can easily understand the other advantages of the present disclosure based on the disclosure of this specification. The present disclosure can also be implemented or applied based on the methods in accordance in different examples. It is possible to modify and/or alter the following examples for carrying out this disclosure without contravening its scope for different aspects and applications.

First of all, all the terms used herein, comprising descriptive and technical terms, are described to have the obvious meaning as commonly understood by one of ordinary skill in the art to which this disclosure belongs. However, the terms may have different meanings according to an intention of one of ordinary skill in the art, case precedents, or the appearance of new technologies. Also, some terms may be arbitrarily selected by the applicant, and in this case, the meaning of the selected terms will be described in detail in the descriptions of the present disclosure. Thus, the terms used herein have to be defined based on the meaning of the terms together with the descriptions throughout the specification.

Also, when a part “comprises” or “includes” a component or a step, unless there is a particular description contrary thereto, the part can further include other components or other steps, not excluding the others.

It is further noted that, as used in this disclosure, the singular forms “a,” “an,” and “the” include plural referents unless expressly and unequivocally limited to one referent. The term “or” is used interchangeably with the term “and/or” unless the context clearly indicates otherwise.

The term “effective amount” used herein in the context refers to an amount of an active ingredient needed to reduce, inhibit or prevent a disease in the subject. It is understood by one of ordinary skilled in the art that the effective amount may change based on the routes of administration, usage of the carriers, and the possibility for combining with other therapeutical methods.

The term “subject” used herein in the context refers to the subjects that may have a liver-related disease, such as hepatitis, abnormal lipid metabolism of liver, abnormal carbohydrate metabolism of liver, liver fibrosis, liver cirrhosis or liver hepatocellular carcinoma, etc. Specifically, the subjects may be for example, animal, particularly vertebrate, for example, but not limit to mammal, particularly human.

The term “a subject in need” used herein in the context refers to the subject that is diagnosed with or may have a liver-related disease, such as hepatitis, abnormal lipid metabolism of liver, abnormal carbohydrate metabolism of liver, liver fibrosis, liver cirrhosis or liver hepatocellular carcinoma etc. Suitable health workers or physicians are able to apply a procedure or an instruction known in the art to identify the subject. The same procedure or instruction known in the art may be also used to determine whether a disease or a symptom is improved in the subject or to determine the most effect amount of the pharmaceutical composition of the present disclosure for administering to the subject.

The terms “improve,” “reduce,” “decrease” or “inhibit” used herein in the context refer to prevent, reduce or decrease the severity or frequency of one or more symptom(s) or abnormality in the subject diagnosed with or may have a liver-related disease, such as hepatitis, abnormal lipid metabolism of liver, abnormal carbohydrate metabolism of liver, liver fibrosis, liver cirrhosis or liver hepatocellular carcinoma, which can be observed by the subject being treated or the third party.

The numeral ranges used herein are inclusive and combinable, any numeral value that falls within the numeral scope herein could be taken as a maximum or minimum value to derive the sub-ranges therefrom. For example, it should be understood that the numeral range “1-60%” comprises any sub-ranges between the minimum value of 1% to the maximum value of 60%, such as the sub-ranges 1% to 40%, 10% to 60%, and 15% to 45%. In addition, a plurality of numeral values used herein can be optionally selected as maximum and minimum values to derive numerical ranges. For instance, the numerical ranges of 1% to 30%, 1% to 60%, or 30% to 60% can be derived from the numeral values of 1%, 30%, and 60%.

Isothiocyanates present in the cruciferous plant, such as broccoli, radish, head cabbage, brassica oleracea plant etc., wherein the isothiocyanates are natural products such as benzyl isothiocyanate (BITC), phenylethyl isothiocyanate (PEITC), allyl isothiocyanate (AITC), phenyl isothiocyanate, sulforaphane (SFN). However, there are still various limitations of isothiocyanates in clinical application, comprising the specific molecular targets, the effective amount and the side effects.

The present disclosure provides a use of isothiocyanate structural modified compound in the manufacture of a pharmaceutical composition for treating or preventing the liver-related disease such as hepatitis, abnormal lipid metabolism of liver, abnormal carbohydrate metabolism of liver, liver fibrosis, liver cirrhosis or liver hepatocellular carcinoma etc. in a subject, and the pharmaceutical composition includes the isothiocyanate structural modified compound and a pharmaceutically acceptable carrier thereof, and the isothiocyanate structural modified compound is

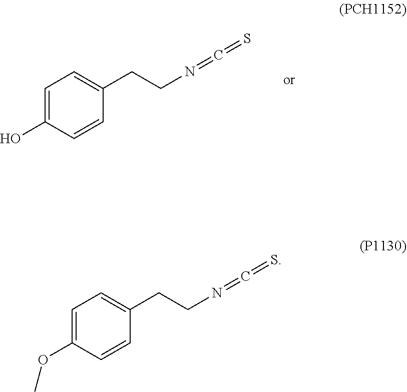

In an embodiment of the present disclosure, the pharmaceutical composition further comprises a pharmaceutically acceptable carrier. In another embodiment of the present disclosure, the pharmaceutically acceptable carrier may be physiologically acceptable excipient or diluent. In still another embodiment of the present disclosure, the examples of the physiologically acceptable excipient or diluent comprise, but not limit to, lactose, starch, dextrin, cyclodextrin, carboxymethyl starch sodium, carboxy starch propionate, microcrystalline cellulose, carboxymethyl cellulose, maltodextrin and magnesium stearate.

In at least one embodiment of the present disclosure, the pharmaceutically acceptable carrier may be filler, binder, preservative, disintegrant, lubricant, suspension, wetting agent, flavoring agent, thickener, acid, biocompatibility solvent, surfactant, complexation reagent or any combination thereof.

In at least one embodiment of the present disclosure, the binder may be paste, sorbitol, guargum, polyvinylpyrrolidone, cellulose derivative such as hydroxypropyl methylcellulose, carboxymethyl cellulose, carbomer (commercially available as Carbopols) or any combination thereof.

In at least one embodiment of the present disclosure, the preservative may be sodium benzoate, methylparaben, propyl paraben, cresol or any combination thereof.

In at least one embodiment of the present disclosure, the lubricant may be metal stearate (e.g. magnesium stearate, calcium stearate or sodium stearate), stearic acid, talcum powder, polyethylene glycol, soluble salt (e.g. sodium chloride or sodium benzoate) or any combination thereof.

In at least one embodiment of the present disclosure, the pharmaceutically acceptable carrier may be polyethylene glycol (PEG), alkylene glycol, propylene glycol, sebacic acid, dimethyl sulfoxide (DMSO), ethanol or any combination thereof. The examples of alkylene glycol are, but not limit to 2-ethyl-1,3-hexanediol and propylene glycol. The example of PEG is, but not limit to PEG-400.

In at least one embodiment of the present disclosure, the amount of the isothiocyanate structural modified compound is 1% to 60% by weight of the pharmaceutical composition. For example, the lower limit of the amount of the isothiocyanate structural modified compound is 1%, 5%, 10%, 15%, 20% or 25% by weight of the composition, and upper limit of the amount of the isothiocyanate structural modified compound is 60%, 55%, 50%, 45%, 40% or 35% by weight of the composition.

In at least one embodiment of the present disclosure, the amount of the pharmaceutically acceptable carrier is 25% to 99% by weight of the composition. For example, the lower limit of the amount of the pharmaceutically acceptable carrier in the composition is 25%, 30%, 35% and 40% by weight of the composition, the upper limit of the amount of the pharmaceutically acceptable carrier in the composition is 99%, 95%, 90%, 80%, 70% and 60% by weight of the composition.

In at least one embodiment of the present disclosure, the pharmaceutically acceptable carrier is at least one selected from the group consisting of: 10 weight% to 40 weight% PEG, 5 weight% to 10 weight% propylene glycol, 1 weight% to 5 weight% sebacic acid, 0 weight% to 15 weight% p-toluenesulfonic acid, 10 weight% to 20 weight% 2-ethyl-1,3-hexanediol, 0 weight% to 10 weight% DMSO, and 0 weight% to 20 weight% ethanol.

In at least one embodiment of the present disclosure, the pharmaceutical composition can be prepared in the form suitable for parenteral administration, intravenous injection, continuous infusion, sublingual administration, subcutaneous administration, topical administration or oral administration. For example, the pharmaceutical composition may be, but not limit to injection, dry powder, oral liquid, wafers, films, tablets, capsules, granules, pills, gels, lotion, ointment, emulsifiers, magma, cream, eye drops or past.

In at least one embodiment of the present disclosure, the pharmaceutical composition may be administered by topical or nebulized means to the subject via intratumor, intravenous, subcutaneous, intracutaneous, oral, intrathecal, intraperitoneal, intranasal, intramuscular, intrapleural administration.

In at least one embodiment of the present disclosure, in some of the specific examples, the active ingredient of the pharmaceutical composition is administered to the subject in the amount of about 0.01 mg to about 10 mg/70 kg body weight per person. In the other embodiment, the active ingredient of the pharmaceutical composition is administered to the subject in the amount of about 1.0 mg to about 5 mg/70 kg body weight per person. In another embodiment, the active ingredient used in the method of the present disclosure is about 10 mg to about 100 mg/70 kg body weight per person, e.g. about 20 mg to about 80 mg/70 kg body weight per person, about 20 mg to about 50 mg/70 kg body weight per person, about 25 mg to about 50 mg/70 kg body weight per person, about 30 mg to about 50 mg/70 kg body weight per person, about 35 mg to about 50 mg/70 kg body weight per person, or about 30 mg to about 40 mg/70 kg body weight per person.

In at least one of the specific embodiments of the present disclosure, the pharmaceutical composition may be administered to the subject once to four times a day, once to four times a week, or once to four times a month. In at least one embodiment of the present disclosure, the pharmaceutical composition may be administered to the subject in a treatment period or cycle of one to four week(s) or one to four month(s). In still some embodiments of the present disclosure, the pharmaceutical composition may be administered to the subject two to three times a week in the period of two-week treatment. In at least one embodiment of the present disclosure, the pharmaceutical composition is administered to the subject four or more times in the first period of the treatment.

The following description further illustrates the effect of the examples in the present disclosure, but is not intended to limit the scope of the present disclosure.

### EXAMPLES

The present disclosure is further illustrated via the following examples. However, the examples are only the instances for the present disclosure, and are not intended to limit the scope and meaning of the present disclosure. In fact, various modifications and changes of the present disclosure are obvious to the person skilled in the art after reading the specification of the present application for carrying out this disclosure without contravening its scope.

### Example 1: PCH1152 and P1130 Inhibit the Cell Growth of the Liver Hepatocellular Carcinoma

The natural isothiocyanate compounds, such as BITC or PEITC, have been proved to inhibit the cell growth and invasion of the liver hepatocellular carcinoma, and they are able to induce apoptosis of the liver hepatocellular carcinoma cell line. However, if BITC or ITC intends to have the inhibiting effect on cancer cells, the dose needed is about at the level of 10 mM, which has limitations in the clinical application due to the difficulty in being developed into drugs. In the present disclosure, the inhibition effects (GI50) of different isothiocyanate structural modified compounds on the growth activities of liver hepatocellular carcinoma cell line SNU-449 and liver hepatocellular carcinoma cell line Mahlavu were examined by MTS analysis. The results show that the structural modified isothiocyanate compounds PCH1152 and P1130 of the present disclosure increased the ability of inhibiting the growth of the liver hepatocellular carcinoma cell line by 12 times as compared to those of the natural isothiocyanate compounds BITC or PEITC, and was also higher than the anticancer drugs Sorafenib and Regorafenib (Table 1).

TABLE 1
The inhibiting effect (GI50) of isothiocyanate compound on the
growth activity of liver hepatocellular carcinoma cell line

| | GI50M, μM | |
|---|---|---|
| | SNU-449 liver | Mahlavu liver |
| Isothiocyanate | hepatocellular carcinoma | hepatocellular carcinoma |
| compound | cell line | cell line |
| BITC | 9.58 | 3.84 |
| PEITC | 7.58 | 9.59 |
| P1130 | 0.64 | 1.52 |
| PCH1152 | 0.89 | 1.54 |
| Sorafenib | 2.34 | 3.58 |
| Regorafenib | 4.54 | 5.00 |

### Example 2: PCH1152 and P1130 Promote the Apoptosis of Liver Hepatocellular Carcinoma Cells

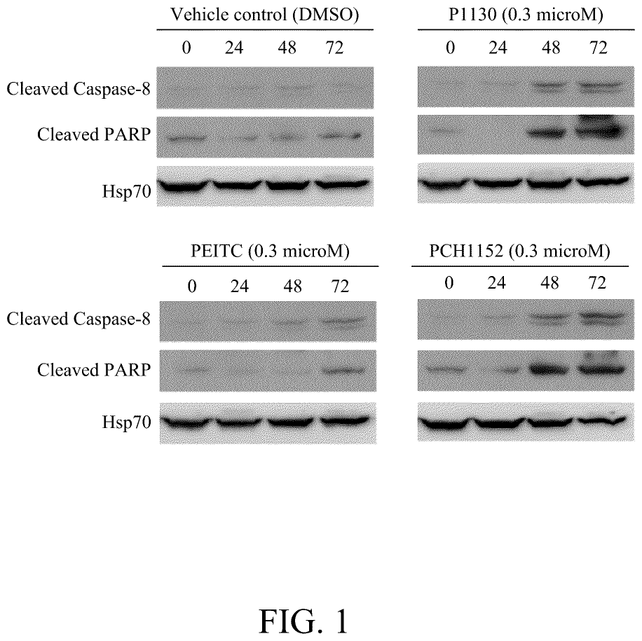

SNU-449 liver hepatocellular carcinoma cell line was treated by 0.3 μM isothiocyanate structural modified compounds P1130 and PCH1152, and natural isothiocyanate compound PEITC, and the control group was treated by DSMO. After extracting the proteins in the cells at different time points, such as 0, 24, 48 and 72 hours, analysis and comparison were performed by using western blot. The results indicate that the expression levels of cleaved caspase 8 and poly (ADP-ribose) polymerase (Cleaved PARP) in P1130- and PCH1152-treated SNU-449 liver hepatocellular carcinoma cell line had significantly increased; in contrast, those in PEITC group had only slightly increased. The results represent that the two structural modified compounds described above effectively promoted the apoptosis of the liver hepatocellular carcinoma cells (FIG. 1).

### Example 3: PCH1152 and P1130 Inhibit the Expressions of Epithelial-Mesenchymal Transition-, Anti-Apoptosis- and Drug Resistance-Related Molecules

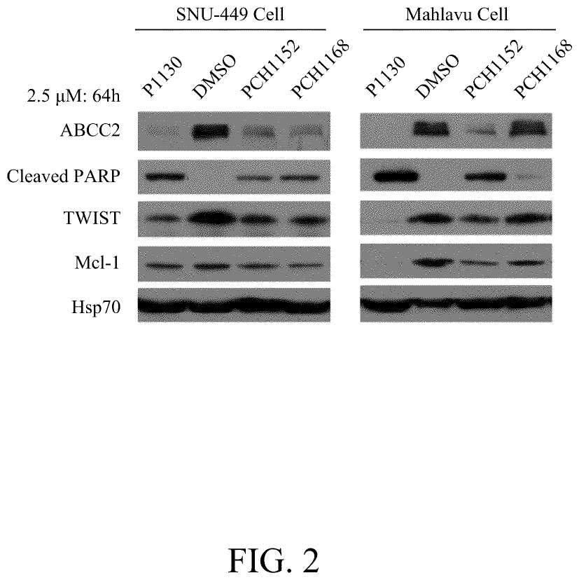

The process of trans-differentiating epithelial cells into motile mesenchymal cells is called epithelial-mesenchymal transition (EMT). EMT is indispensable in development, wound healing and stem cell behavior, and may induce fibrosis and carcinogenesis pathologically. In addition, EMT plays an important role in liver fibrosis, liver cirrhosis and liver hepatocellular carcinoma. The overexpression of the EMT regulatory proteins Twist and Snail is related to the development of liver hepatocellular carcinoma, it also leads to a higher malignancy, e.g. invasiveness and metastasis, of the liver hepatocellular carcinoma cells, and can inhibit the expressions of epithelial markers such as E-cadherin, and increase the expressions of mesenchymal markers such as N-cadherin. Moreover, EMT may increase the anti-apoptosis effect, which will result in the development of drug resistance in cancer treatment, and induce the development of stemness of the cancer cells, as well as increase the number of cancer stem cells. The cancer stem cell is one of the important factors in the progression, recurrence, metastasis and drug resistance of the cancer. There is an evidence that in the samples of the liver hepatocellular carcinoma patients with poor prognosis, the overexpressions of Twist and Snail were observed. SNU-449 and Mahlavu liver hepatocellular carcinoma cell lines were treated by 2.5 μM of isothiocyanate structural modified compounds P1130 and PCH1152, separately in this experiment, and the inhibition of the expressions of all multidrug resistance-associated protein ABCC2, EMT regulatory protein Twist and anti-apoptosis protein Mcl-1 were observed after 64 hours (FIG. 2). The results above represent that isothiocyanate structural modified compound of the present disclosure may prevent liver carcinogenesis and the growth, invasion, metastasis, progression and recurrence of liver hepatocellular carcinoma via inhibiting epithelial-mesenchymal transition, anti-apoptosis and/or inhibiting drug resistance.

### Example 4: PCH1152 and P1130 Have Selectivity to Liver Hepatocellular Carcinoma Cell, and Can Inhibit the Expressions of the Liver Disease/Liver Hepatocellular Carcinoma-Related Molecules ABCC2, Mcl-1 and Twist

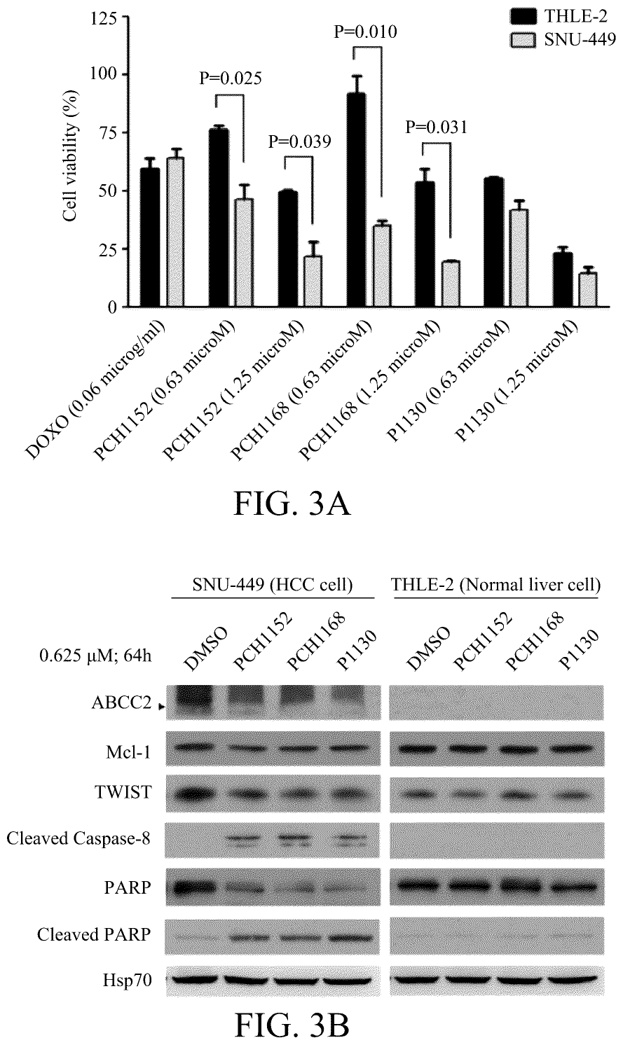

Preferable small molecule anticancer drugs must have higher selectivity to cancer cells, thereby reducing the toxicity and side effects in the normal cells and the dosage being taken. The selectivity of isothiocyanate structural modified compound of the present disclosure to liver hepatocellular carcinoma cells was examined by using human normal liver cell line THLE-2 and liver hepatocellular carcinoma cell line SNU-449. After treating liver hepatocellular carcinoma cell line and normal liver cell line by isothiocyanate structural modified compounds P1130 or PCH1152, the cell growth was evaluated by MTS assay. The results show that the structural modified compound PCH1152 substantially decreased the cell viability of the liver hepatocellular carcinoma cell line group; by contrast, the effect of PCH1152 was not significant in the human normal liver cell line group, and the effects in the two groups described above were significantly different (p=0.025) (FIG. 3A). These results show that PCH1152 had a better cell growth inhibition selectivity to liver hepatocellular carcinoma. In addition, this result also shows that conventional anticancer drug Doxorubicin did not have selectivity between normal liver cell and liver hepatocellular carcinoma cell.

Furthermore, western blot analysis shows that isothiocyanate structural modified compounds PCH1152, P1130 and PCH1168 at low concentration may promote apoptosis of liver hepatocellular carcinoma cell line, and inhibit the expressions of liver disease- and liver hepatocellular carcinoma-related molecules, comprising drug resistance-related protein ABCC2, anti-apoptosis protein Mcl-1 and EMT regulatory molecule Twist; by contrast, there was no such effect in the human normal liver cell line (FIG. 3B). These results also show that isothiocyanate structural modified compound PCH1152, P1130 and PCH1168 of the present disclosure had higher cell growth inhibition selectivity to liver hepatocellular carcinoma.

### Example 5: PCH1152 Inhibits the Growth of the Mouse Liver Hepatocellular Carcinoma; Maintains the Normal Function of Liver; and Inhibits the Expressions of the Liver Disease/Liver Hepatocellular Carcinoma-Related Molecules ABCC1, USP22 and Mcl-1

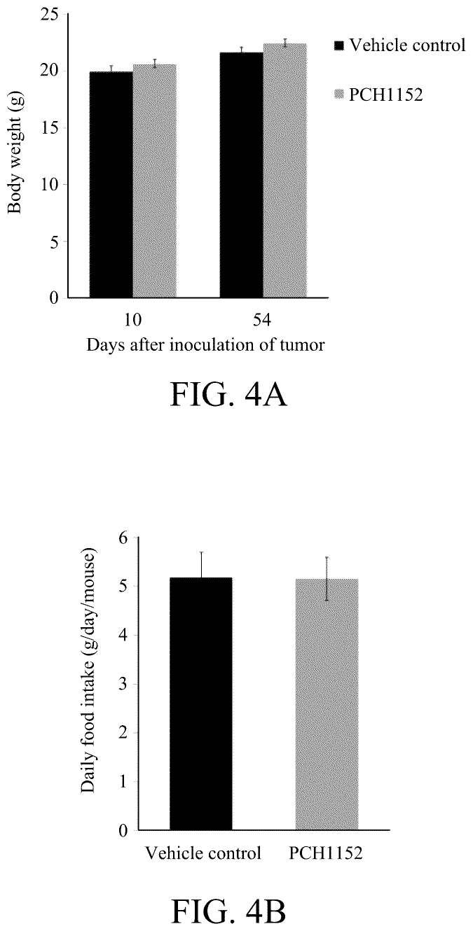

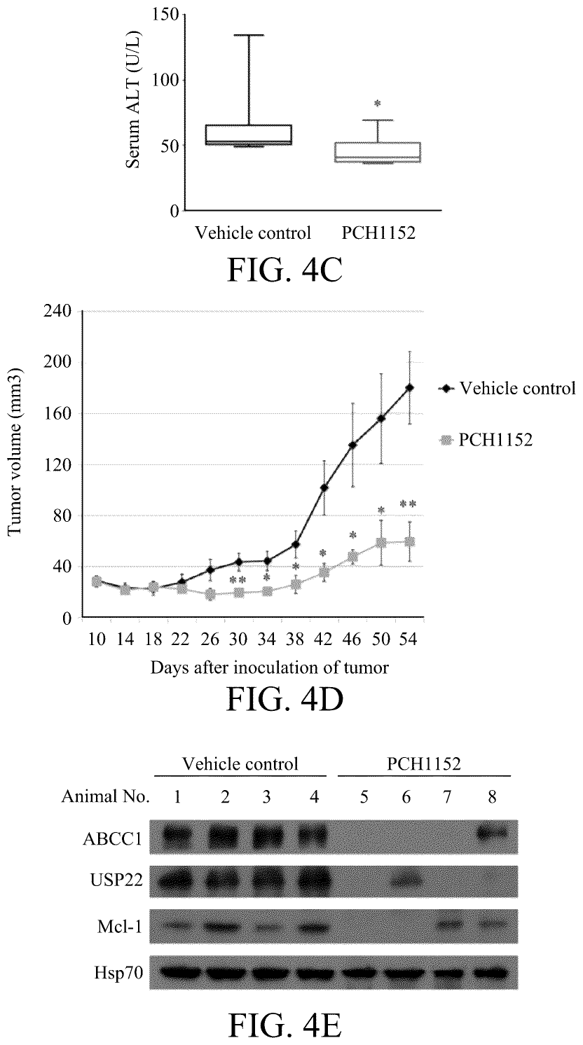

In order to prove that isothiocyanate structural modified compound PCH1152 has an inhibiting effect on the growth of the tumor in an animal's body, a mouse animal model of xenograft model was applied as an animal disease model. Seven days after subcutaneously injecting 1×10 6 Mahlavu liver hepatocellular carcinoma cell line into nude mice, PCH1152 (0.6 mg/mouse/day) began to be orally administered to the mice. After continuously administrating the drug orally for 45 days, the mice were sacrificed and their subcutaneous tumor were taken and analyzed. No mouse dead during the experiment, and the experimental record shows that PCH1152 did not have any significant effect on the mouse weight (FIG. 4A) and daily food intake (FIG. 4B). In addition, PCH1152 was able to decrease the value of ALT in the mouse (FIG. 4C), indicating that PCH1152 may have a protective effect on the liver. The subcutaneous tumor volume (length×width²×0.5236) of the mice were measured and recorded regularly every two days, the mice that orally administered with PCH1152 were observed to have significantly smaller subcutaneous tumor volume (FIG. 4D). Furthermore, the protein expressions in the subcutaneous tumor of the mice were analyzed by western blot, and it was found that the expressions of the liver hepatocellular carcinoma-related molecules, such as drug resistance-related protein ABCC1, ubiquitin-specific protease USP22, anti-apoptosis protein Mcl-1, of the mice orally administered with PCH1152 were significant lower (FIG. 4E).

The results above show that continuous administering PCH1152 orally to the mouse liver hepatocellular carcinoma xenograft model at a dosage of 0.6 mg/mouse/day is able to maintain the normal function of liver, to have a protective effect on liver, to inhibit the growth of the subcutaneous tumor, and to reduce the expression of the liver hepatocellular carcinoma-related protein; and a possible mechanism of the inhibiting effect on the tumor is by inhibiting the liver hepatocellular carcinoma-related molecules ABCC1, USP22, and Mcl-1.

### Example 6: PCH1152 Removes Reactive Oxygen Species (ROS)

Reactive oxygen species (ROS) signal transduction pathway may induce abnormal metabolism of lipid, and lead to accumulation of lipid in the liver, thereby resulting in fatty liver disease and hepatitis. Accumulation of reactive oxygen species will also lead to liver injury and activate the process of the liver fibrosis; excessive reactive oxygen species has a critical effect on the development of the liver hepatocellular carcinoma and drug resistance of the liver hepatocellular carcinoma.

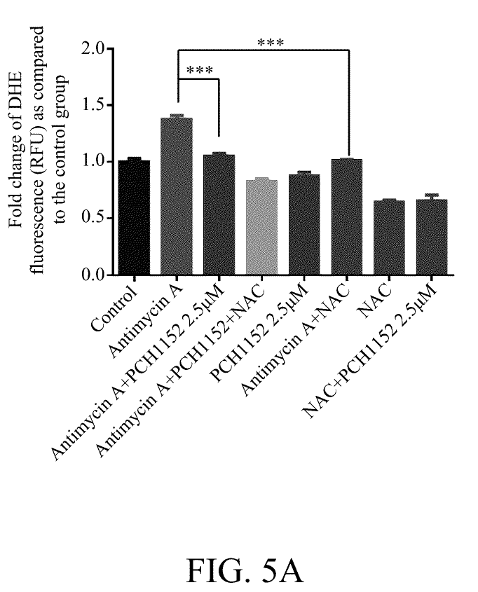

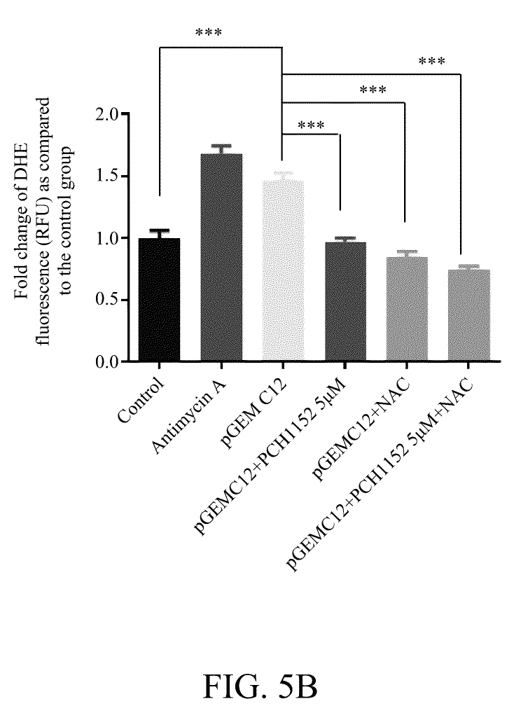

In order to prove the ability of PCH1152 in removing ROS, the LX-2 cells were treated with 2.5 μM PCH1152, or 2.5 μM PCH 1152 in combination with NAC (N-acetylcysteine, which is effective in removing ROS) under the condition with or without Antimycin A (for producing ROS) for 24 hours to examine the effect of removing ROS by PCH1152 alone or PCH1152 combined with NAC. ROS was examined by DHE fluorescence, and the difference between each group was compared by Tukey's Multiple Comparison Test. The results show that PCH1152 may significantly reduce the Antimycin A-induced ROS increasement, in addition, treatment of PCH1152 in combination with NAC may more significantly reduce the Antimycin A-induced ROS increasement as compared to the treatment of PCH1152 or NAC alone, indicating that PCH1152 or NAC has a synergistic effect on removing ROS (FIG. 5A and Table 2A).

TABLE 2A
The effect of PCH1152 and NAC on removing ROS.

| | Significant |
|---|---|
| | (*p < 0.05, |
| | **p < 0.01, |
| Comparing groups | ***p < 0.001) |
| Control vs Antimycin A | *** |
| Control vs Antimycin A + PCH1152 2.5 μM | ns |
| Control vs Antimycin A + PCH1152 + NAC | * |
| Control vs PCH1152 2.5 μM | ns |
| Control vs Antimycin A + NAC | ns |
| Control vs NAC | *** |
| Control vs NAC + PCH1152 2.5 μM | *** |
| Antimycin A vs Antimycin A + PCH1152 2.5 μM | *** |
| Antimycin A vs Antimycin A + PCH1152 + NAC | *** |
| Antimycin A vs PCH1152 2.5 μM | *** |
| Antimycin A vs Antimycin A + NAC | *** |
| Antimycin A vs NAC | *** |
| Antimycin A vs NAC + PCH1152 2.5 μM | *** |
| Antimycin A + PCH1152 2.5 μM vs Antimycin A + | ** |
| PCH1152 + NAC | |
| Antimycin A + PCH1152 2.5 μM vs PCH1152 2.5 μM | * |
| Antimycin A + PCH1152 2.5 μM vs Antimycin | ns |
| A + NAC | |
| Antimycin A + PCH1152 2.5 μM vs NAC | *** |
| Antimycin A + PCH1152 2.5 μM vs NAC + PCH1152 | *** |
| 2.5 μM | |
| Antimycin A + PCH1152 + NAC vs PCH1152 2.5 μM | ns |
| Antimycin A + PCH1152 + NAC vs Antimycin | * |
| A + NAC | |
| Antimycin A + PCH1152 + NAC vs NAC | * |
| Antimycin A + PCH1152 + NAC vs NAC + PCH1152 | * |
| 2.5 μM | |
| PCH1152 2.5 μM vs Antimycin A + NAC | ns |
| PCH1152 2.5 μM vs NAC | ** |
| PCH1152 2.5 μM vs NAC + PCH1152 2.5 μM | ** |
| Antimycin A + NAC vs NAC | *** |
| Antimycin A + NAC vs NAC + PCH1152 2.5 μM | *** |
| NAC vs NAC + PCH1152 2.5 μM | ns |

In addition, in order to prove that PCH1152 has an ability in removing Hepatitis B virus (HBV)-induced ROS, human liver hepatocellular carcinoma cell line Huh-7 was transfected with the expression plasmid (pGEM C12) of Hepatitis B virus (HBV) strain C12 for 48 hours to induce the development of ROS. To examine the effect of PCH1152, NAC or the combination thereof on removing HBV-induced ROS, the Huh-7 cells were remained untreated, or treated with 5 μM PCH1152, 10 μM NAC or 5 μM PCH 1152 plus 10 μM NAC. The ROS was examined by DHE fluorescence, and the difference between each group was compared by Tukey's Multiple Comparison Test. The results show that both PCH1152 and NAC can significantly reduce the HBV-induced ROS increasement, in addition, treatment of PCH1152 in combination with NAC may more significantly reduce the HBV-induced ROS increasement as compared to the treatment of PCH1152 or NAC alone, indicating that PCH1152 or NAC has a synergistic effect on removing HBV-induced ROS (FIG. 5B and Table 2B).

TABLE 2B
The effect of PCH1152 and NAC on removing HBV-induced ROS.

| | Significant |
|---|---|
| | (*p < 0.05, |
| | **p < 0.01, |
| Comparing groups | ***p < 0.001) |
| Control vs pGEM C12 | *** |
| Control vs pGEM C12 + PCH1152 5 μM | ns |
| Control vs pGEM C12 + NAC | ns |
| Control vs pGEM C12 + PCH1152 5 μM + NAC | * |
| Control vs NAC + PCH1152 2.5 μM | *** |
| pGEM C12 vs pGEM C12 + PCH1152 5 μM | *** |
| pGEM C12 vs pGEM C12 + NAC | *** |
| pGEM C12 vs pGEM C12 + PCH1152 5 μM + NAC | *** |

### Example 7: PCH1152 Inhibits the Incidence Rate of the Liver Hepatocellular Carcinoma in Hepatitis B Mouse, Inhibits the Growth of the Tumor, and Alleviates the Abnormality of Serum Liver Function Index

In order to further prove the inhibiting effect of long-term usage of PCH1152 on the incidence and growth of liver tumor, the Hepatitis B virus (HBV) transgenic mouse model with high incidence of tumor was used for the test (see U.S. patent application Ser. No. 17/127,496; invention title: ANIMAL MODEL FOR HEPATOCELLULAR CARCINOMA AND USES THEREOF). To examine the incidence rate of mouse liver tumor under long-term PCH1152 administration, PCH1152 was added into the drinking water of the mice when the mice were three-month-old, and the drinking water was replaced every 7 to 14 days. The termination of the experiment was set at 19 months, and the blood of the mice started to be collected every 2 to 3 months when the mice were 9-month-old, and the ALT values of the serum were measured. When the ALT of the serum were higher than 150 U/L twice in a row, the mice were sacrificed and the developments of the liver tumors thereof were observed.

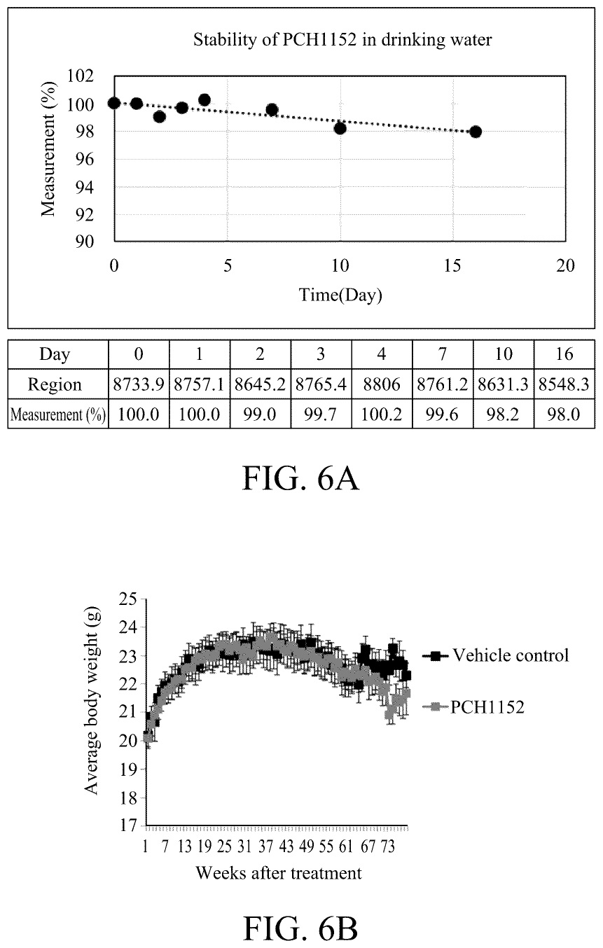

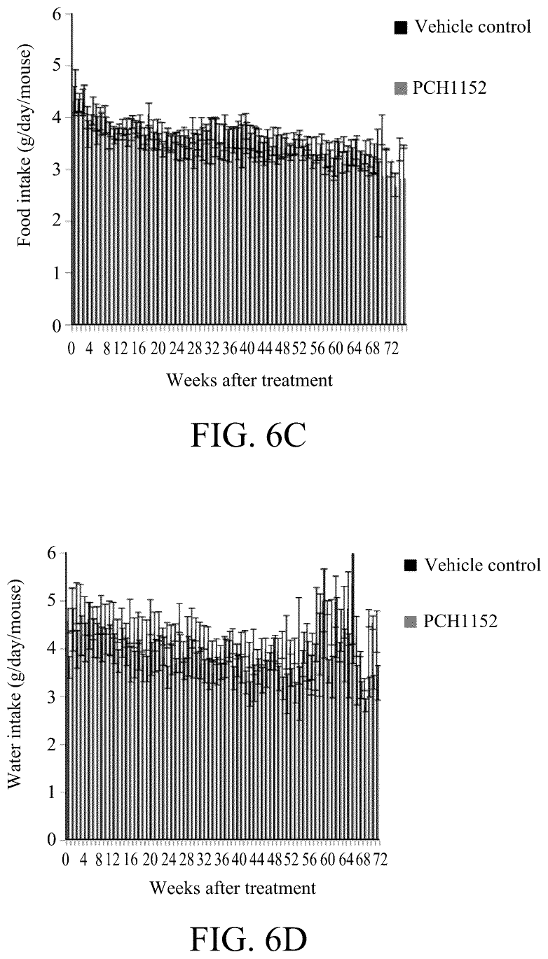

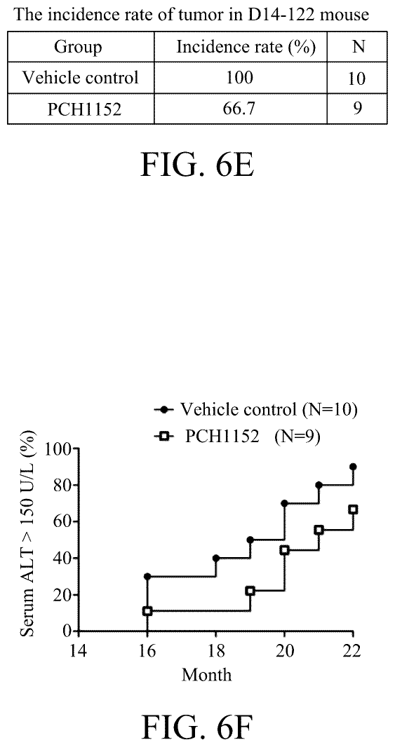

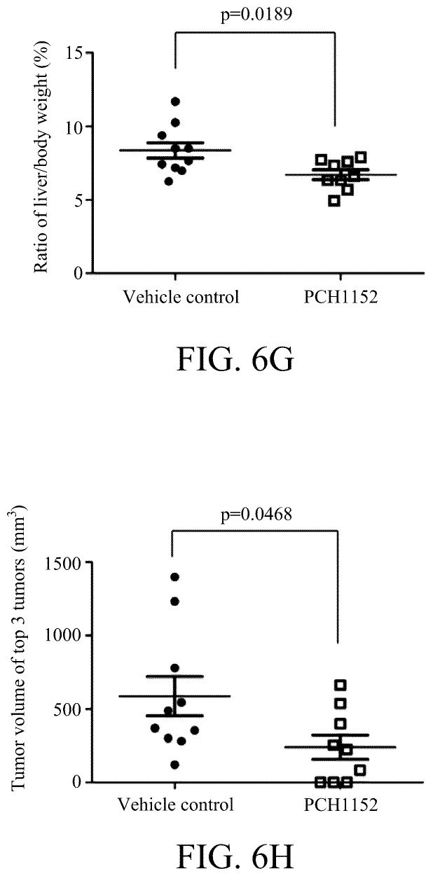

The results show that the structural stability of the PCH1152 was still higher than 98% after being added in the drinking water of the mice for 16 days (FIG. 6A); and long-term treatment of PCH1152 did not have any significant impact on the weight (FIG. 6B), daily food intake (FIG. 6C), and daily water intake (FIG. 6D) of the Hepatitis B virus (HBV) transgenic mouse.

The results also show that long-term treatment of PCH1152 may reduce the incidence rate of liver tumor for more than 30% as compared to that of the control group (100% vs. 66.7%) (FIG. 6E and Table 3). Moreover, PCH1152 may defer the increasement of alanine aminotransferase (ALT) in the mice. The results show that in 50% of the mice of the PCH1152 treatment group, the serum ALT thereof increased to over 150 U/L at 21-month old, which is older than that of the control group (19.5-month old) (FIG. 6F). PCH1152 may also significantly decrease the weight (FIG. 6G) and the volume (FIG. 6H) of the liver tumor.

TABLE 3
The incidence rate of the liver hepatocellular
carcinoma in Hepatitis B mouse.

| | Control group (N = 10) | PCH1152 (N = 9) | P-value |
|---|---|---|---|
| The incidence | 100% (10/10) | 66.7% (6/9) | 0.0466 |
| rate of liver | | | |
| hepatocellular | | | |
| carcinoma | | | |

### Example 8: PCH1152 Reduces Hepatic Lipid Accumulation and Liver Fibrosis

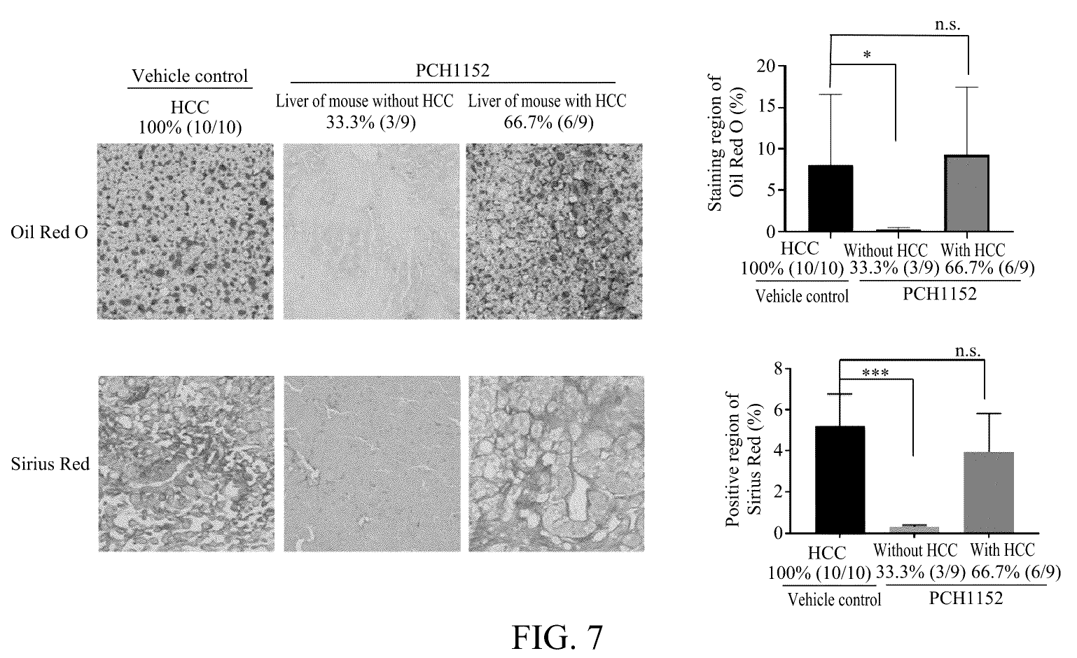

In order to demonstrate the effect of PCH1152 against hepatic lipid accumulation or fibrosis, the liver tissues of the mice in Example 7 were stained with Oil Red and Sirius Red; the results show that as compared to that of the control group, either the region of the lipid accumulation or fibrosis were observed to be reduced significantly in the liver tissues of the mice treated with PCH1152 and without developing liver hepatocellular carcinoma (FIG. 7).

### Example 9: PCH1152 Enhances the Autophagy

Autophagy protects the liver cells from injury and death by eliminating the injured organelles and proteins in the patients with liver-related diseases; by contrast, abnormal autophagy may be the pathway for promoting the development and progression of liver disease.

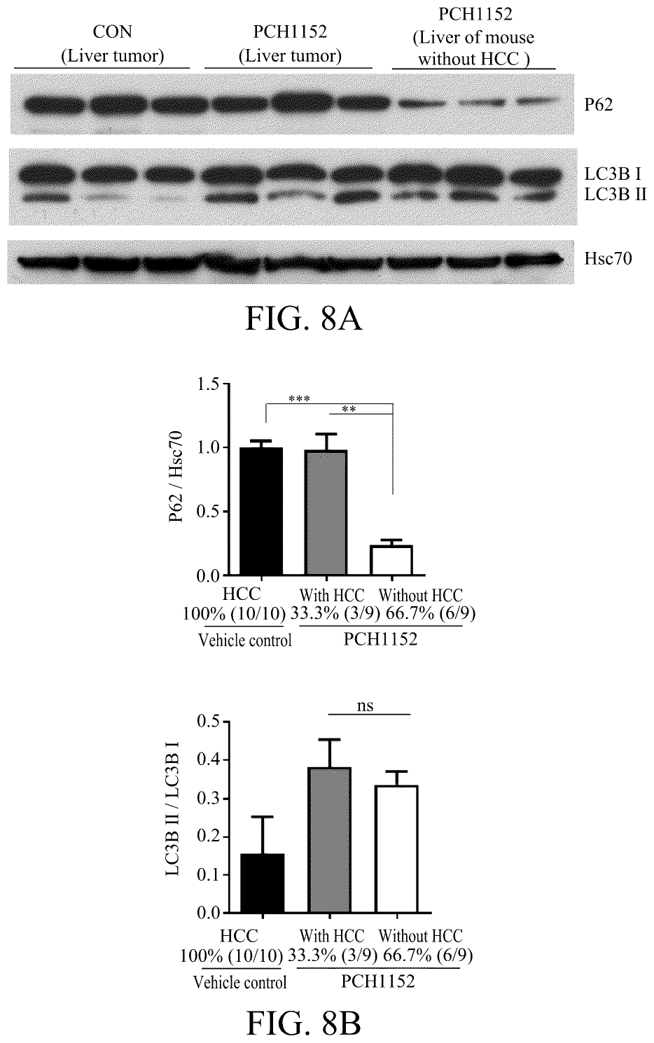

The liver tissues of the mice with or without liver hepatocellular carcinoma in the example 7 were analyzed by western blot (FIG. 8A), and quantitative analysis was performed via Image J software (FIG. 8B). P62 protein accumulated in the cells when the autophagy was forbidden, and it was degraded when the autophagy started-up. Therefore, P62 protein may be a biomarker of autophagy. The liver tissue of the mouse treated with PCH1152 and without developing liver hepatocellular carcinoma had a significantly lower P62 protein as compared to the mouse developing liver hepatocellular carcinoma (the ratio of P62 and Hsc70 was lower; the amount of Hsc70 protein was used as the internal control), indicating that PCH1152 may significantly enhance autophagy, thereby leading to the degradation of the P62 protein (FIG. 8B). Moreover, LC3B protein is the subunit of the microtubule-related protein, which has different protein isomers. After the autophagy being started, LC3B I isomer will be transformed into LC3B II isomer. FIG. 8B shows that the ratio of LC3B II and LC3B I in the PCH1152 group was higher than that in the control group, and PCH1152 did not affect the starting-up ability of autophagy. The results above indicate that PCH1152 may enhance autophagy, i.e. may inhibit abnormal “anti-autophagy” effect, thereby preventing the development of liver hepatocellular carcinoma. **p<0.01; ***p<0.001; unpaired t-test.

### Example 10: PCH1152 and Anticancer Drug Have Synergistic Effect

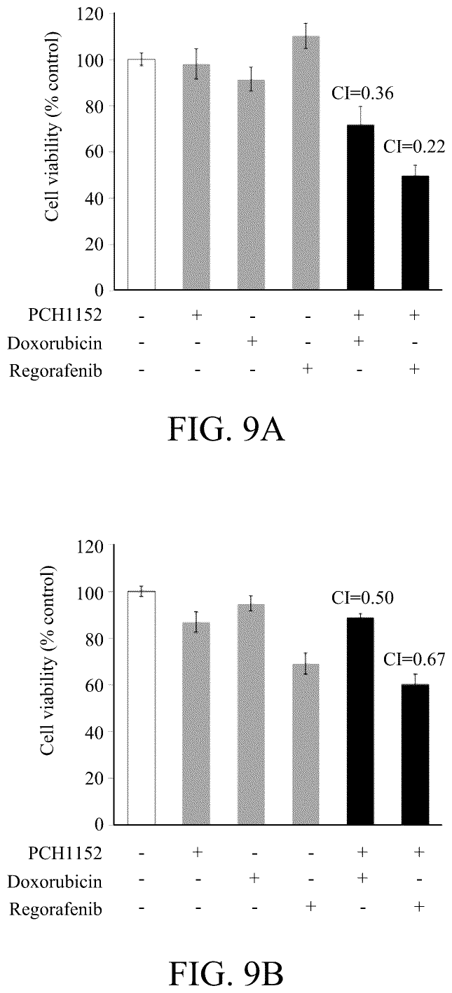

In the cancer therapy, it is believed that using a combination of multiple drugs is more effective than using only one drug. To evaluate the synergistic effect of the drugs, the combinations of PCH1152 (0.25 μM) and anticancer drug Doxorubicin (0.0125 μg/mL) or Regorafenib (1.25 μM) were used in the SNU-449 and Mahlavu liver hepatocellular carcinoma cell line, and the combination indexes (CI) were calculated by Calcusyn software (Biosoft, UK). CI<1 represents that the drugs have an additive property. The results show that when PCH1152 was used in combination with Doxorubicin or Regorafenib, in either Mahlavu liver hepatocellular carcinoma cell line (FIG. 9A) or SNU-449 liver hepatocellular carcinoma cell line (FIG. 9B), the CI values were lower than 1, indicating that PCH1152 had the synergistic effect in inhibiting the cell growth of liver hepatocellular carcinoma when being combined with Doxorubicin or Regorafenib, respectively.

### Example 11: PCH1152 Has a Synergistic Effect with Metabolic Disease Drug, Cardiovascular Disease Drug or Endocrine Disease Drug

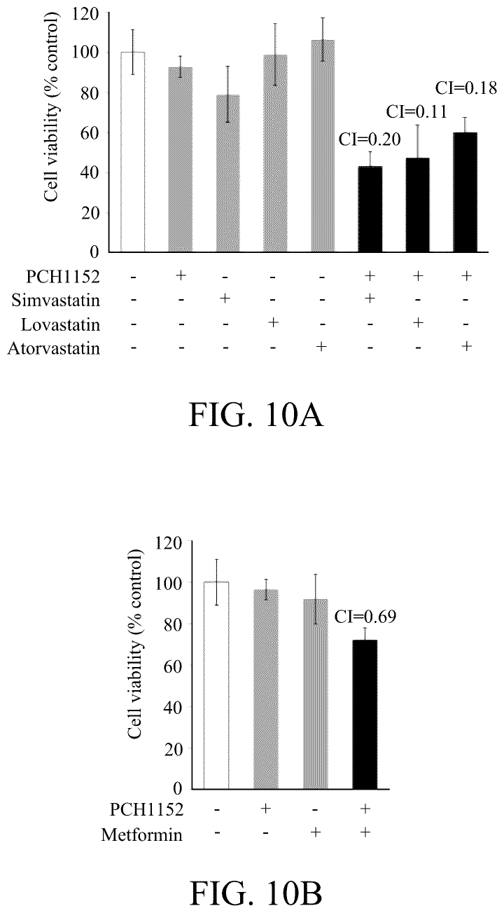

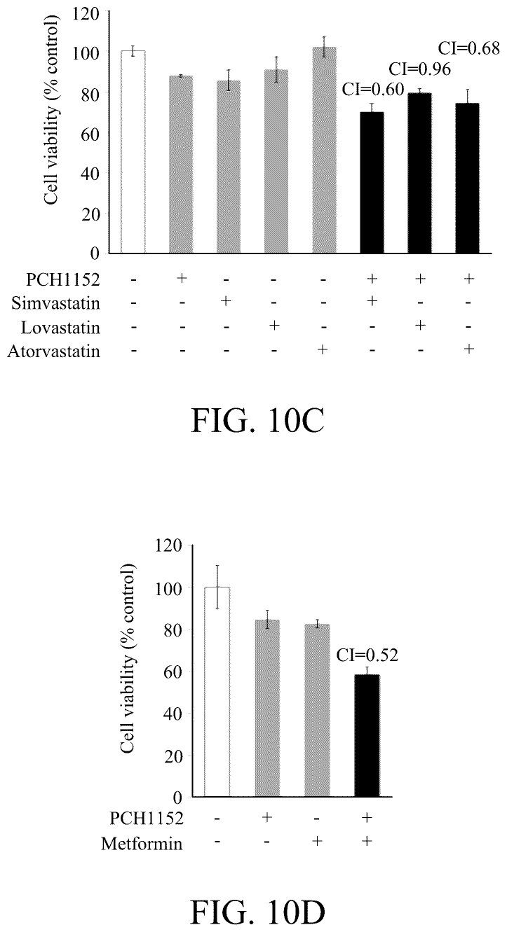

0.25 μM PCH1152 in combination with 1 μM statin hypolipidemic agent (Simvastatin, Lovastatin, Atorvastatin) or 2.5 mM hypoglycemic agent (Metformin) were used to evaluate their synergistic effects by CI value, wherein CI<1 represents that the drugs have additive property. The results show that using PCH1152 in combination with Simvastatin, Lovastatin, Atrovastatin or Metformin, respectively, had synergistic effect in inhibiting the growth of the tumor cells in Mahlavu liver hepatocellular carcinoma cell line (FIG. 10A and FIG. 10B) and SNU-449 liver hepatocellular carcinoma cell line (FIG. 10C and FIG. 10D).

### Example 12: Establishment of the Sorafenib-Drug Resistance-Liver Hepatocellular Carcinoma Cell Line

In order to look for the biomarkers of drug resistance for liver hepatocellular carcinoma, liver hepatocellular carcinoma cell line Hep3B and SNU-449 were continually cultured in an environment with Sorafenib, and the concentration of Sorafenib was increased gradually. Specifically, broth containing 3.5 μM to 10.8 μM Sorafenib was used as a starting concentration (compared to the IC50 of the cell), and the concentration increased 0.5 μM every week until the Sorafenib-drug resistance-liver hepatocellular carcinoma cell line was established. By applying MTS assay to test the cell viability, it was found that Sorafenib-drug resistance-cell line had a higher IC50 as compared to the IC50 of wild-type liver hepatocellular carcinoma cell line, indicating that the Sorafenib-drug resistance-cell line had a higher resistance to the Sorafenib-induced cell death (Table 4); moreover, the structure analog of Nexavar (Sorafenib), Stivarga (Regorafenib), has been approved as a second-line therapy for patients using Sorafenib. With the same experimental procedures as described above, the Sorafenib-drug resistance-cell line was also cultured in a cell broth containing Regorafenib, and the cell viability was tested by MTS assay. The results show that Sorafenib-drug resistance-cell line also had a higher resistance to the Regorafenib-induced cell death (Table 5).

TABLE 4
The half-maximal inhibitory concentration (IC50)
of Sorafenib in Sorafenib-drug resistance-
liver hepatocellular carcinoma cell line

| liver | The IC50 of Sorafenib, μM | |
|---|---|---|
| hepatocellular | wild-type liver | drug resistance-liver |
| carcinoma cell | hepatocellular carcinoma | hepatocellular carcinoma |
| line | cell line | cell line |
| SNU-499 | 7.89 | 14.22 |
| Hep3B | 3.80 | 10.93 |

TABLE 5
The half-maximal inhibitory concentration (IC50) of Regorafenib in
Sorafenib-drug resistance-liver hepatocellular carcinoma cell line

| liver | The IC50 of Regorafenib, μM | |
|---|---|---|
| hepatocellular | wild-type liver | drug resistance-liver |
| carcinoma cell | hepatocellular carcinoma | hepatocellular carcinoma |
| line | cell line | cell line |
| SNU-499 | 3.66 | 14.50 |
| Hep3B | 4.28 | 8.33 |

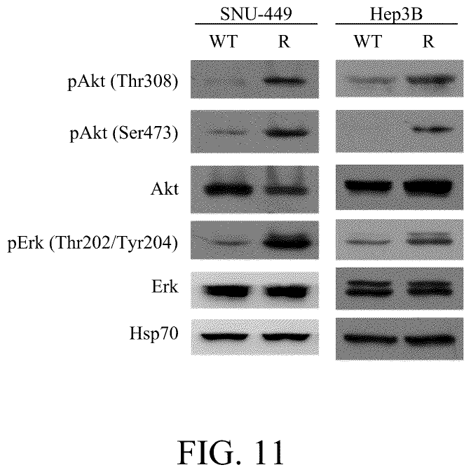

Previous studies disclosed various mechanisms of the development of Sorafenib drug resistance in liver hepatocellular carcinoma, such as increasing the activity of Akt/Erk signal transduction pathway. Accordingly, the established Sorafenib-drug resistance-liver hepatocellular carcinoma cell line was analyzed by western blot with Akt specific antibody (Anti-phospho-Akt/Akt antibodies; 1:1000; #2965/#4060/#4691; Cell Signaling Technology, Inc.) and Erk specific antibody (Anti-phospho-Erk/Erk antibodies; 1:1000; #4370/#4695; Cell Signaling Technology, Inc.). The results show that both the expressions of phosphorylated Akt and phosphorylated Erk were increased in the Sorafenib-drug resistance-liver hepatocellular carcinoma cell line, proving that the Sorafenib-drug resistance-liver hepatocellular carcinoma cell line was successfully established (FIG. 11).

### Example 13: Increasment of the Expressions of Ubiquitin-Specific Protease USP22 and Multidrug Resistance-Associated Protein ABCC1 in Sorafenib-Drug Resistance-Liver Hepatocellular Carcinoma Cell Line

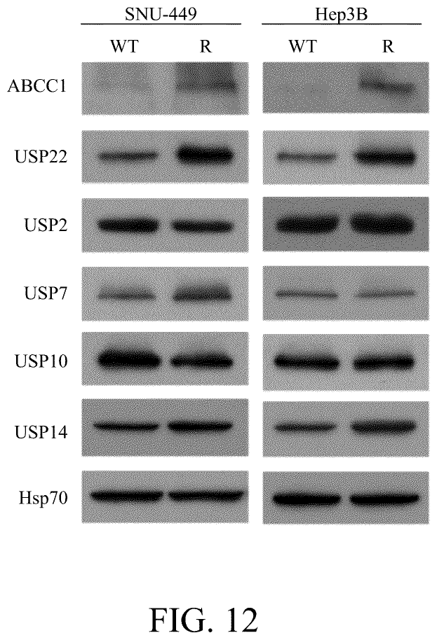

Ubiquitin modification system participates in multiple important regulations of cell physiology. Ubiquitin-specific protease (USPs) is the largest group in the Deubiquitinating protease family (DUBs), which has been known to have 56 members in human cells. The abnormal expression of USP has a high correlation with the cancers occurrence and progression, and may be used to predict the post-diagnostic tumor recurrence, metastasis and lower viability, therefore, it may be a new target for cancer therapy. For example, overexpression of USP7 is correlated to the malignancy of liver hepatocellular carcinoma. The expression of USP14 is increased significantly in the tumor tissue of liver hepatocellular carcinoma, and USP14 is involved in the mechanisms leading to the progression of the tumor. The high expression of USP22 is also observed in the patients with liver hepatocellular carcinoma, wherein USP22 mediates the multiple drug resistance of liver hepatocellular carcinoma by SIRT1/AKT/ABCC1 signaling pathway; while decreasing the expression of USP22 enhances the sensitivity of liver hepatocellular carcinoma cell line to anticancer drug. Liver hepatocellular carcinoma cell line with Sorafenib drug resistance was analyzed and examined by western blot with USPs specific antibody (Anti-USP antibody sampler Kit; 1:2000; #12894; Cell Signaling Technology, Inc.), USP22 specific antibody (Anti-USP22 antibodies; 1:2000; ab195289; Abcam, Cambridge, MA, USA) and ABCC1 specific antibody (Anti-ABCC1 antibody; 1:2000; #72202; Cell Signaling Technology, Inc.). The results show that the protein expression levels of USP22 and ABCC1 were increased in liver hepatocellular carcinoma cell line with Sorafenib drug resistance (FIG. 12).

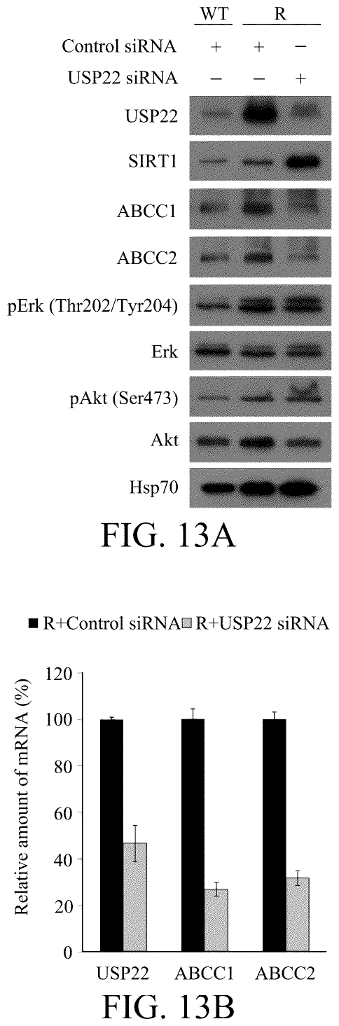

Previous literature indicates that USP22 may increase the expression of ABCC1 via activating Akt signaling pathway (Ling, S., et al., USP22 mediates the multidrug resistance of hepatocellular carcinoma via the SIRT1/AKT/MRP1 signaling pathway. Mol Oncol, 2017. 11(6): p. 682-695). In the liver hepatocellular carcinoma cell line with Sorafenib drug resistance, using RNA interference technology to inhibit the endogenous cellular expression of USP22 may also promote decreasements of the protein expression levels (FIG. 13A) and mRNAs (FIG. 13B) of ABCC1 and ABCC2. The results show that the increasements of the expression levels of ABCC1 and ABCC2 were mediated by USP22 in the liver hepatocellular carcinoma cell line with Sorafenib drug resistance. That is, USP22 plays an important role in Sorafenib drug resistance.

### Example 14: PCH1130 or PCH1152 Reverses the Drug Resistance of the Liver Hepatocellular Carcinoma

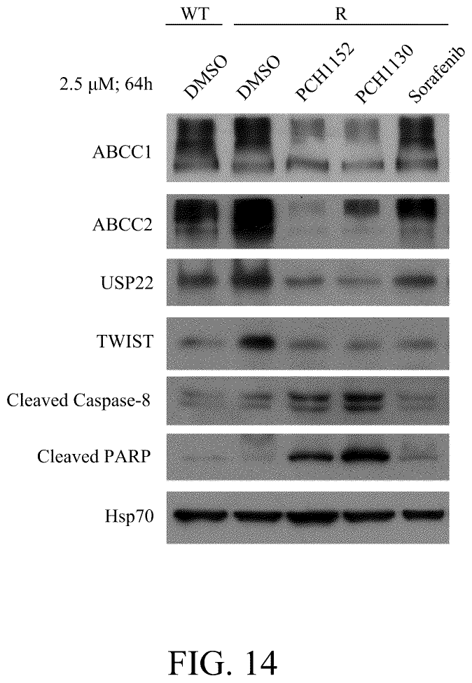

After treating liver hepatocellular carcinoma cell line with Nexavar drug resistance by PCH1130 or PCH1152, the expressions of molecules such as USP22, ABCC1, ABCC2 and Twist in liver hepatocellular carcinoma may be decreased, and the expressions of cleaved Caspase-8 and PARP may be increased, which induced the apoptosis of liver hepatocellular carcinoma cells. The results show that PCH1130 or PCH1152 had the effect on reversing the drug resistance in liver hepatocellular carcinoma (FIG. 14).

### Example 15: The Protein Expressions of USP22 and ABCC1 in Liver Hepatocellular Carcinoma with Sorafenib Drug Resistance Shows Positive Correlation Between Each Other

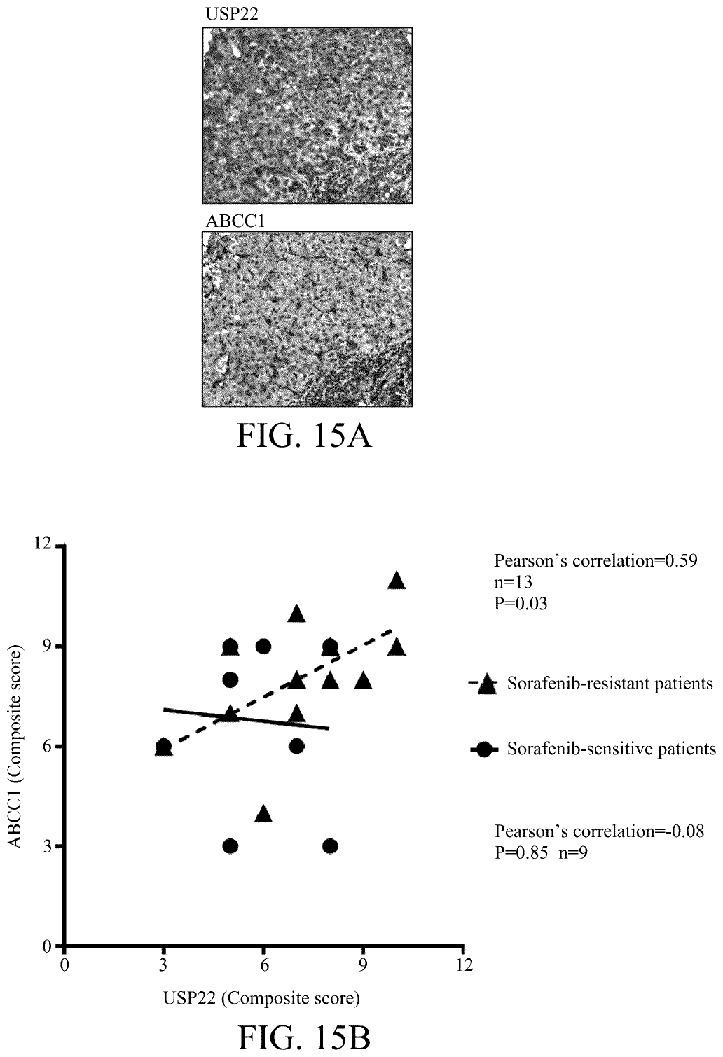

In 13 human liver hepatocellular carcinoma patients with Sorafenib drug resistance and 9 human liver hepatocellular carcinoma patients with Sorafenib sensitivity, the expressions of USP22 and ABCC1 in liver hepatocellular carcinoma tissues removed by surgery were analyzed by immunohistochemistry staining. The results show that the expressions of USP22 and ABCC1 were detected in the liver hepatocellular carcinoma tissues of patients with Sorafenib drug resistance (FIG. 15A), furthermore, the protein expression levels of USP22 and ABCC1 showed positive correlation between each other (FIG. 15B); by contrast, the protein expressions of USP22 and ABCC1 did not show significant correlation in liver hepatocellular carcinoma of patients with Sorafenib sensitivity. The above results show that examination of the protein expressions of USP22 and ABCC1 in patients with liver hepatocellular carcinoma is suitable for taking as the biomarkers for screening Sorafenib-drug resistance-liver hepatocellular carcinoma.

### Example 16: Safety Test

The mouse xenograft model of liver hepatocellular carcinoma was administered with different concentrations of PCH1152 or P1130 via intravenous injection, and the median lethal dose (LD50) was calculated by the number of death and dosage. The results show that the toxicity of PCH1152 or P1130 was comparable to the toxicity of existing anticancer drug, e.g. Taxotere (Table 6).

TABLE 6
The LD50 of PCH1152 and P1130

| | LD50 (mg/kg) |
|---|---|
| P1130 | 100 < LD50 < 300 |
| PCH1152 | 30 < LD50 < 100 |

### CONCLUSION

The isothiocyanate structural modified compound of the present disclosure demonstrates the following unexpected effects: having a stronger cell growth inhibiting ability to liver hepatocellular carcinoma by 12 times than that of natural isothiocyanate compound, and also having a significant effects on promoting the apoptosis of liver hepatocellular carcinoma cells; inhibiting reactive oxygen species; inhibiting the expressions of epithelial-mesenchymal transition regulatory protein Twist, anti-apoptosis protein Mcl-1, multidrug resistance-associated protein ABCC1, multidrug resistance-associated protein ABCC2, and ubiquitin-specific protease USP22; enhancing autophagy; decreasing the accumulation of lipid in liver; and reducing the level of liver fibrosis. Moreover, the isothiocyanate structural modified compounds do not have significant effect on human normal liver cell line, showing that it has selectivity to liver hepatocellular carcinoma cell. On the other hand, the isothiocyanate structural modified compounds have synergistic effect on inhibiting the growth of liver hepatocellular carcinoma when being used in combination with anticancer drug; the isothiocyanate structural modified compounds also show synergistic effect when being used in combination with metabolic disease drug, cardiovascular disease drug or endocrine disease drug. In the mouse disease model, it also proves that the isothiocyanate structural modified compound of the present disclosure has a significant inhibiting effect on the growth of the liver tumor. In addition, USP22 and ABCC1 are highly expressed in liver hepatocellular carcinoma cell line with Sorafenib drug resistance, and show positive correlation between each other, which is suitable for taking as biomarkers of Sorafenib drug resistance. The isothiocyanate structural modified compound can reverse the drug resistance, which can also effectively inhibit the growth of liver hepatocellular carcinoma and the expressions of liver hepatocellular carcinoma-related molecules in liver hepatocellular carcinoma cell line with Sorafenib drug resistance.

The above description of the specific examples is merely to illustrate the content of the present disclosure. However, the scope of the present disclosure does not limit to the examples disclosed above. It is understood that the specification and the examples of the present application are intended to be exemplary; therefore, the scope of the claims of the present application should be given the widest explanation, which covers all the modifications and alterations in accordance with the principle of the present disclosure.

What is claimed is:

1. A method for preventing or treating a liver disease, comprising administering a therapeutically effective amount of pharmaceutical composition to a subject in need, wherein the pharmaceutical composition comprises an isothiocyanate structural modified compound and a pharmaceutically acceptable carrier thereof.

2. The method of claim 1, wherein the isothiocyanate structural modified compound is

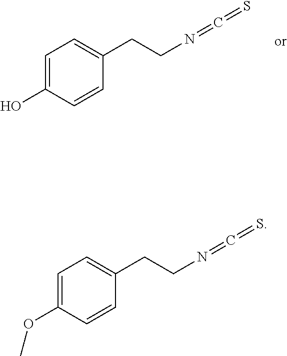

3. The method of claim 1, wherein the liver disease is selected from the group consisting of hepatitis, abnormal lipid metabolism of liver, abnormal carbohydrate metabolism of liver, liver fibrosis, liver cirrhosis and liver hepatocellular carcinoma or any combination thereof.

4. The method of claim 3, wherein the liver hepatocellular carcinoma is liver hepatocellular carcinoma with drug resistance to an anticancer drug, and the anticancer drug comprises Sorafenib (Nexavar), Doxorubicin (Adriamycin) or Regorafenib (Stivarga).

5. The method of claim 3, wherein the liver hepatocellular carcinoma is liver hepatocellular carcinoma with drug resistance to an anticancer drug, and the drug resistance is evaluated by taking expressions of ubiquitin-specific protease USP22 and multidrug resistance-associated protein ABCC1 in the subject as biomarkers, and the expression of USP22 and the expression of ABCC1 show positive correlation between each other.

6. The method of claim 1, wherein the cause of the liver disease is selected from the group consisting of epithelial-mesenchymal transition (EMT), reactive oxygen species (ROS) accumulation, abnormal autophagy and anti-apoptosis or any combination thereof.

7. The method of claim 1, wherein the liver disease is liver hepatocellular carcinoma, and the prevention or treatment comprises inhibiting the occurrence, growth, invasion, metastasis, progression, recurrence or drug resistance of the liver hepatocellular carcinoma.

8. The method of claim 1, wherein the liver disease is liver hepatocellular carcinoma, and the prevention or treatment comprises reversing drug resistance of the liver hepatocellular carcinoma or increasing the sensitivity of the liver hepatocellular carcinoma to an anti-liver hepatocellular carcinoma drug.

9. The method of claim 1, wherein the prevention or treatment comprises maintaining normal function of the liver.

10. The method of claim 1, wherein the prevention or treatment comprises inhibiting the expression level of the liver disease-related molecule, wherein the liver disease-related molecule is selected from the group consisting of reactive oxygen species, multidrug resistance-associated protein ABCC1, multidrug resistance-associated protein ABCC2, EMT regulatory molecule Snail, EMT regulatory molecule Twist, anti-apoptosis molecule Mcl-1, ubiquitin-specific protease USP22 or any combination thereof.

11. The method of claim 1, wherein the subject is a human.

12. The method of claim 1, wherein the pharmaceutical composition is administered orally, intravenously, enterally or subcutaneously.

13. The method of claim 1, wherein the pharmaceutical composition is administered in combination with an anticancer drug.

14. The method of claim 13, wherein the anticancer drug comprises targeted therapy drug, immunotherapy drug or radiotherapy drug.

15. The method of claim 13, wherein the anticancer drug comprises Sorafenib (Nexavar), Doxorubicin (Adriamycin) or Regorafenib (Stivarga).

16. The method of claim 13, wherein the pharmaceutical composition is administered in combination with the anticancer drug simultaneously, sequentially, or separately.

17. The method of claim 1, wherein the pharmaceutical composition is administered in combination with a metabolic disease drug, a cardiovascular disease drug or an endocrine disease drug.

18. The method of claim 17, wherein the metabolic disease drug, cardiovascular disease drug or endocrine disease drug comprises drugs for blood glucose control or blood lipid control.

19. The method of claim 17, wherein the metabolic disease drug, cardiovascular disease drug or endocrine disease drug comprises Metformin or Statin-like drug.

20. The method of claim 17, wherein the pharmaceutical composition is administered in combination with the metabolic disease drug, cardiovascular disease drug or endocrine disease drug simultaneously, sequentially, or separately.
